(19) United States
(12) Patent Application Publication
(10) Pub. No.: US 2026/0346184 A1
(43) Pub. Date: Nov. 5, 2026

(54) NITROGEN REMOVAL SYSTEM FOR METHANE PURIFICATION FROM LANDFILL GAS, AND METHOD THEREOF

(71) Applicant: Archaea Energy Inc., Houston, TX (US)

(72) Inventor: Michael J. Mitariten, Pittstown, NJ (US)

(21) Appl. No.: 19/100,078

(22) PCT Filed: Aug. 8, 2023
(86) PCT No.: PCT/US2023/071867

§ 371 Date: Jan. 30, 2025

Related U.S. Application Data
(60) Provisional application No. 63/576,452, filed on Aug. 9, 2022.

(51) Int. Cl. B01D 53/047; C07C 7/13; C10L 3/08; C10L 3/10

(52) U.S. Cl. CPC B01D 53/047; C07C 7/13; C10L 3/08; C10L 3/105; B01D 2253/102; B01D 2253/1085; B01D 2256/245; B01D 2257/102; B01D 2259/402; C10L 2290/12; C10L 2290/26; C10L 2290/46; C10L 2290/542

(57) ABSTRACT

The present invention is directed towards a system and a method that produce a product stream of methane of pipeline quality containing at least 95% by volume of methane from a landfill gas feed, comprising two sequential nitrogen rejection PSA units (NRUs) each of which contains a methane specific absorbent, wherein the adsorbed methane within the second PSA unit is desorbed, recovered and recycled back to the feed inlet for the first PSA unit to enrich the product stream produced from the first NRU.

### FIELD OF THE INVENTION

This invention relates to the purification of landfill gas, and, more particularly, to the removal of nitrogen from landfill gas by the use of a methane selective adsorbent in a novel pressure swing adsorption (“PSA”) process to produce pipeline quality methane.

### BACKGROUND OF THE INVENTION

The removal of nitrogen from landfill gas is of considerable importance inasmuch as nitrogen is present in a significant extent. Nitrogen contamination lowers the heating value of the natural gas and increases the transportation cost based on unit heating value.

Applications that are aimed to remove nitrogen and other impurities from natural gas streams provide significant benefits to the U.S. economy. In 1993, the Gas Research Institute (GRI) estimated that 10-15% (~22 trillion cubic feet) of the natural gas reserves in the U.S. are defined as sub-quality due to contamination with nitrogen, carbon dioxide, sulfur. Most of these reserves, however, have discounted market potential, if they are marketable at all, due to the inability to cost effectively remove the nitrogen. Nitrogen and carbon dioxide are inert gases with no BTU value and must be removed to low levels (4% by volume typically) before the gas can be sold.

Unlike crude oil, it is not economical to bring imports of natural gas into North America, therefore pricing of natural gas could be expected to rise forcing end users to seek alternative fuels, such as oil and coal, that are not as clean burning as gas. While base consumption for natural gas in the U.S. is projected to grow, one segment may grow much more rapidly. Natural gas usage in electric power generation is expected to grow rapidly because natural gas is efficient and cleaner burning allowing utilities to reduce emissions.

Concurrently, due to the rising population, warfare conflicts, and/or delay of infrastructure upgrades to alternative heating sources, the worldwide demand for natural gas has increased.

Further, capturing methane reduce emissions of greenhouse gas, which is shorter-lived but far more potent than carbon dioxide. In 2021 alone, according to U.S. Environmental Protection Agency, number of renewable natural-gas projects in the U.S. surged from 125 to 174. The Coalition for Renewable Natural Gas, which counts Waste Management, Chevron Corp., Shell PLC and Duke Energy among its members, says the renewable methane fuel accounts for just 0.03% of the U.S. gas market, but could cover 75% of current U.S. residential demand or 45% of industrial demand by 2040. Therefore, there is a need to develop a cost-effective method to upgrade currently unmarketable sub-quality reserves and renewable gas in the U.S., thereby increasing the proven reserve inventory.

Methods heretofore known for purification of natural gas, in particular, nitrogen removal, may be divided roughly into five classifications:
(a) Methods involving fractional distillation at low temperature and (usually) high pressure, i.e. cryogenics. Since nitrogen has a lower boiling point than methane and the other hydrocarbons present in natural gas, it may be removed as a gas on liquefying the remaining constituents which are then revaporized.
(b) By selective adsorption of the methane and higher hydrocarbons on an adsorbent such as activated charcoal. The adsorbed gases are then desorbed to yield a gas reduced in of nitrogen.
(c) Miscellaneous processes involving selective diffusion through a series of organic membranes, formation of lithium nitride by treatment with lithium amalgam, absorption of the nitrogen in liquid ammonia or in liquid sulphur dioxide.
(d) Pressure swing adsorption (“PSA”) system that uses nitrogen specific adsorbent to remove nitrogen.
(e) Membrane separation technology.

The principal disadvantage of the fractional distillation and adsorption processes is that they remove the major component, methane, from the minor component, nitrogen, instead of the reverse. In cryogenic processing, almost the entire volume of natural gas must be refrigerated, usually compressed, and then heated again. Accordingly, cryogenic processing is expensive to install and operate, limiting its application to a small segment of reserves. Cryogenic technology is believed only capable of cost effectively purifying reserves, which exceed 50,000,000 standard cubic feet of gas per day and as well having nitrogen contamination levels of 15% by volume or more. Gas reserves that do not fit these criteria are not currently being purified. The potential value of this gas is totally lost as the wells are usually capped. The processes suggested under paragraph (c) above are handicapped by an unsatisfactory degree of separation or by the use of very expensive materials. Many routes to use cryogenic distillation exists and one example is assigned to Waga Energy U.S. Pat. No. 10,905,995.

In smaller-scale natural gas operations as well as in other areas such as synthesis gas and coke oven gas processing, adsorption processes can be especially well suited. The adsorption capacities of adsorption units can, in many cases, be readily adapted to process gas mixtures of varying nitrogen content without equipment modifications, i.e. by adjusting adsorption cycle times. Moreover, adsorption units can be conveniently skid-mounted, thus providing easy mobility between gas processing locations. Further, adsorption processes are often desirable because more than one component can be removed from the gas. As noted above, nitrogen-containing gases often contain other gases that contain molecules having smaller molecular dimensions than nitrogen, e.g., for natural gas, carbon dioxide, oxygen and water, and for coke oven gas, carbon monoxide.

In the case of PSA, the lost of methane can be a substantial economic penalty because it normally routed to flare and/or atmosphere representing a loss of revenue from the valuable methane/hydrocarbons. This loss of methane can typically range from 1 to 15% by volume.

For the PSA technology there are two approaches. In the first an adsorbent that adsorbs the methane is used with the nitrogen passing through the bed. This technology is well established and a typical patent of the approach is assigned to Honeywell UOP LLC, U.S. Pat. No. 5,174,796.

The second PSA approach is one that uses an adsorbent that adsorbs the nitrogen. This approach is taught by Engelhard U.S. Pat. No. 6,444,012. Similar systems are known to utilize PSA to remove nitrogen.

U.S. Pat. No. 6,290,751 to Regil et al. teaches a method and a system to purify a starting feed containing methane and nitrogen, comprising sending the feed to a first adsorption zone A1 that adsorbs and produces a purified stream of methane, and sending a stream rich in nitrogen from A1 to a second adsorption zone A2 that adsorbs and produces methane and expels nitrogen, wherein both A1 and A2 are comprised of molecular sieves. The methane stream from A2 is desorbed and a fraction of this stream is recycled back to A2, wherein the expelled nitrogen is used to flush A1. Regil et al. does not teach or suggest recycling of methane stream from A2 back to the starting feed A1, and the recycling of the expelled nitrogen is not useful and adds costs to the system.

PCT WO20141 81 860 assigned to Tayo Nippon Sanso Corporation discloses an PSA apparatus containing two or more adsorption columns to recover highly pure nitrogen gas and methane for use in power generation, by separating methane and nitrogen from a source gas using adsorbent having greater equilibrium absorption of methane than of nitrogen. '860 does not teach or suggest recycling of methane stream from columns back to the starting feed to enrich the starting feed, and the means to achieve a high rejection rate of nitrogen for the sole focus of methane pipeline gas production adds additional costs.

U.S. Pat. No. 6,631,626 to Hahn teaches a means to liquefy natural gas wherein a feed going into a molecular sieve for adsorbing nitrogen to recover methane-rich product, then the waste stream is sent to a secondary nitrogen removal carbon unit that adsorbs methane. From the second unit the N₂ is expelled, the methane is being compressed and sent back to be liquified. Hahn focuses on liquifying natural gas utilizing nitrogen adsorbing adsorbent.

U.S. Pub. No. 2021/0229027 to Da Silva Barcia et al. teaches a multi-stage process to remove contaminant gases from biogas by utilizing at least two pressure swing adsorption stages, wherein nitrogen and oxygen are removed in bulk in the first stage, using selective adsorbents, and a nitrogen and oxygen—depleted intermediate stream is yielded to the second stage. The second stage employs an adsorbent or adsorbents to selectively remove carbon dioxide and trace amounts of remaining nitrogen and oxygen, thus producing a purer methane stream. Da Silva Barcia et al. utilizes PSA in both stages to adsorb nitrogen and other gas elements.

U.S. Pat. No. 6,315,817 to Butwell et al. teaches a pressure swing adsorption process to separable hydrogen from a mixture of the same with methane, utilizing two separate PSA stages, one containing a nitrogen selective crystalline zeolite, and the second containing a methane selective adsorbent, in order to generate high purity N₂ gas as a product gas, and purge methane. Butwell et al. achieves the opposite of the objective of the current invention.

U.S. Pat. No. 6,610,124 to Dolan et al. teaches a pressure swing adsorption process to separate impurities such as nitrogen and carbon dioxide, as well as recovery of heavy hydrocarbons from a natural gas stream that utilizes two separate adsorption systems. The first separation adsorption system contains an adsorbent selective for nitrogen, carbon dioxide or both, and the second system contains a heavy hydrocarbon-selective adsorbent, wherein the second system produces a combined stream that is comprised of methane and C₃₊ hydrocarbons. Dolan's system requires a separate C₃₊ hydrocarbon, thereby increasing costs.

The membrane approach is described in a patent assigned to Air Liquide U.S. Pat. No. 10,780,392.

It is the primary objective of this invention to provide a commercially viable PSA process for removing nitrogen from landfill gas.

A further object of the invention is to provide a PSA process for removing nitrogen from landfill gas which can provide a highly concentrated methane product at high process efficiencies.

Another object of this invention is to separate nitrogen from pretreated landfill gas by a novel PSA process which yields a high quality methane product, and purge the nitrogen stream.

Still another object of the invention is to provide and maintain peak efficiency of the methane-selective adsorbent during PSA separation of methane from pretreated landfill gas.

### SUMMARY OF THE INVENTION

The present invention is directed towards a pressure swing adsorption method to produce a high purity methane product stream from a pretreated landfill feed, comprising;
1) Sending a pretreated landfill feed containing at least 80% by volume of methane to a first nitrogen rejection unit (“NRU”) containing a methane selective material to adsorb methane and to produce a purged stream;
2) Desorbing the methane within the first NRU in a PSA cycle to produce a product stream of methane;
3) Sending the purged stream to a second NRU containing a methane selective material to adsorb methane, and to produce a second purged stream;
4) Desorbing the methane from the second NRU in a PSA cycle to be recycled back to the pretreated landfill feed for the first NRU, to enrich the product stream to contain at least 95% by volume of methane, preferably at least 97% by volume of methane.

The present invention is also directed towards a pressure swing adsorption system to produce a high purity methane product stream from a pretreated landfill feed comprising;
1) A first nitrogen rejection unit that contains a methane selective material and is in fluid communication with a pretreated landfill gas feed, such that the first NRU adsorbs methane and allows nitrogen to pass through to produce a purged stream, and that the adsorbed methane is then desorbed and recovered as a product stream to be removed in a separate outlet;
2) A second nitrogen rejection unit that contains a methane selective material and is in fluid communication with the first NRU to receive the first purged waste stream, such that the second NRU adsorbs methane, and allows nitrogen to pass through, to produce a second purged stream; wherein the second NRU is also in separated fluid communication with the pretreated landfill feed inlet to said first NRU, such that the adsorbed methane within the second NRU is desorbed, recovered and sent to the first NRU together with said pretreated landfill gas.

### BRIEF DESCRIPTION OF THE DRAWINGS

FIG. 1 is a schematic view of a system and a method to produce a pipeline stream of methane from a landfill gas feed comprising two nitrogen rejection units.

### DETAILED DESCRIPTION OF THE INVENTION

The present invention is directed towards a pressure swing adsorption method to produce a high purity methane product stream from a pretreated landfill feed, comprising;
1) Sending a pretreated landfill feed containing at least 70% by volume of methane to a first nitrogen rejection unit (“NRU”) containing a methane selective material to adsorb methane and to produce a purged stream containing less than 40% by volume of methane;
2) Desorbing the methane within the first NRU in a PSA cycle to produce a product stream of methane;
3) Sending the purged stream to a second NRU containing a methane selective material to adsorb methane, and to produce a second purged stream of nitrogen that contains less than 20% by volume, preferably less than 10% by volume of methane;
4) Desorbing the methane from the second NRU in a PSA cycle to be recycled back to the pretreated landfill feed for the first NRU, wherein the recycled methane stream contains no more than 70% by volume of methane, to enrich the product stream to contain at least 95% by volume of methane, preferably at least 97% by volume of methane.

The present invention is also directed towards a pressure swing adsorption system to produce a high purity methane product stream from a pretreated landfill feed comprising;
1) A first nitrogen rejection unit that contains a methane selective material and is in fluid communication with a pretreated landfill gas feed, such that the first NRU adsorbs methane and allows nitrogen to pass through to produce a purged stream, and that the adsorbed methane is then desorbed and recovered as a product stream to be removed in a separate outlet;
2) A second nitrogen rejection unit that contains a methane selective material and is in fluid communication with the first NRU to receive the first purged waste stream, such that the second NRU adsorbs methane, and allows nitrogen and other hydrocarbons to pass through, to produce a second purged stream; wherein the second NRU is also in separated fluid communication with the pretreated landfill feed inlet to said first NRU, such that the adsorbed methane within the second NRU is desorbed, recovered and sent to the first NRU together with said pretreated landfill gas.

As is known in the prior art, natural gas streams frequently contain components smaller than nitrogen, such as water vapor, carbon dioxide and hydrogen sulfide. The gas stream to be treated in accordance with the novel process of this invention preferably have these contaminants removed prior to treatment of the feed gas stream in accordance with the novel process of this invention. Methods and systems to pretreated landfill gas to remove these contaminants are well known in the art. Specifically, the landfill gas for the present invention is first treated and produced at 100 psig, to contain about 70% by volume of methane; less than 2% by volume, preferably less than 1% by volume of water vapor; less than 10% by volume, preferably less than 2% by volume of carbon dioxide; and less than below 1,000 ppm, preferably less than 10 ppm by volume of H₂S.

The amount of nitrogen present in the landfill feed gas stream is not critical in carrying out the novel process of this invention and can be as low as 5 mol percent to as high as about 35 mol percent. Typically, the nitrogen content is in the range of 5 to 20 mol percent.

The first NRU unit at first cycle achieves at least 85% by volume recovery of the methane from desorbing and recovering the methane within the methane selective adsorbent, and the % by volume will increase to at least a pipeline quality of 95% by volume of methane, after further enrichment of the feed gas mixed with the desorbed methane from the second NRU. The purged stream from the first NRU will produce a rejected nitrogen rich stream with about 15 to 50% by volume CH₄ and 20% to 50% by volume N₂. The concentration of the rejected nitrogen is dependent on the amount of nitrogen in the landfill gas and the recovery rate of methane in the first stage NRU. A co-current depressurization step is introduced into the first NRU and the second NRU units to desorb methane.

The first purged stream is optionally compressed and sent to the second NRU that contains a CH₄ selective adsorbent to treat the first purged stream to adsorb the CH₄, while letting the nitrogen pass through the adsorbent bed to be purged. Unlike the first NRU unit, this second NRU, or High Recovery NRU Module (“NRM”) does not aim to produce pipeline quality gas, rather it enriches methane to a increased purity at less than pipeline quality (enriched as compared to the NRU reject stream). The adsorbed CH₄ in the second NRU is then regenerated in a PSA cycle, wherein the CH₄ is desorbed at about of 80% by volume. The desorbed CH₄ is optionally compressed, and recycled back to the feed for the first NRU, to achieve a product stream of CH₄ of at least 95%, preferably at least 97% by volume from the first NRU. The N₂ in the purged feed stream from the first NRU passes through the bed of the CH₄ selective adsorbent in the second NRU and becomes a final rejected or purged nitrogen stream, containing less than 10, preferably less than 5% by volume of methane.

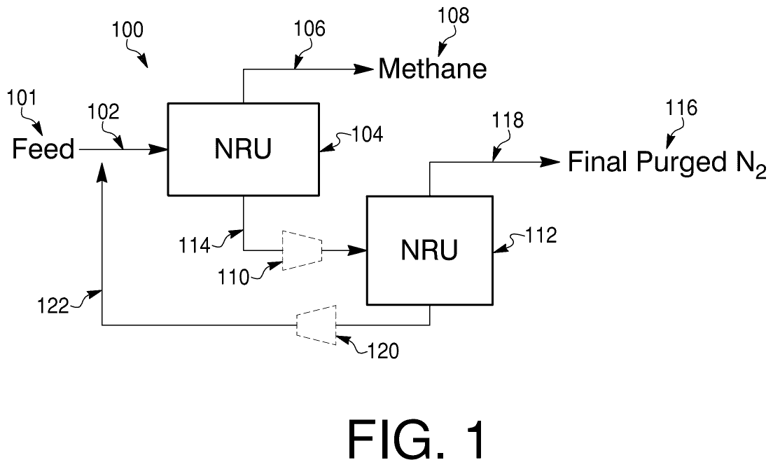

As illustrated in FIG. 1, a nitrogen removal system 100 is operable to remove nitrogen from a methane-containing pretreated landfill feed 101 entering through a feed conduit 102. The feed from 102 enters a first NRU adsorption unit 104 that contains a methane selective bed. Methane is then desorbed, recovered, and sent out of unit 104 as a product stream 108 via a product conduit 106. Product stream 108 contains at least 90% by volume methane, preferably over 95% by volume methane and less than 10%, preferably less than 5% by volume of nitrogen and carbon dioxide.

A nitrogen rich, low methane purged stream is produced from unit 104, and this purged stream is optionally compressed via compressor 110 and send to a second nitrogen rejection unit 112 via conduit 114, wherein the second nitrogen rejection module 112 contains a methane selective adsorbent that adsorbs methane, purges a purified N₂ waste stream 116 from the second NRU 112 unit via conduit 118. The final purged nitrogen waste stream contains at least 75% by volume, preferably at least 90% by volume of nitrogen. A recycled methane stream is desorbed and recovered from the adsorbent bed of the second NRU, optionally compressed in a compressor 120, and recycled to feed conduit 102 via recycled conduit 122. The recycled methane stream contains no more than 70% by volume of methane.

The methane selective adsorbent employed in adsorption units 104 and 112 are made from the same or similar methane adsorbing material, such that the adsorbents have characteristics in which methane has an equilibrium adsorption amount greater than that of nitrogen. Thus, units 104 and 112 adsorb methane preferentially over nitrogen. Various types of adsorbents can be used: activated carbon, zeolite such as crystalline aluminosilicate zeolite (e.g., 13X) or a high aluminum X zeolite having a silicon-to-aluminum ratio of about one or an amorphous adsorbent (e.g., silica gel or carbon), or the like having an average pore diameter of 4.5~15 Å as determined by the MP method and an adsorbed quantity of methane at a temperature of 298 K of 20 Ncc/g or greater at atmospheric pressure can be used. Applicable adsorbent is selected from the group consisting of molecular sieved activated carbon (“MSC”), crushed activated carbon (“AC-1”), granular activated carbon (“AC-2”), pelleted activated carbon and mixtures thereof.

The adsorption performed in adsorption units 104 and 112 can be performed by virtually any known adsorption process such as, for example, pressure swing adsorption (PSA), thermal swing, displacement purge, or nonabsorbable purge (i.e., partial pressure reduction). However, the process of the present invention can be advantageously performed using a pressure swing cycle. Pressure swing cycles are well known in the art. It is preferred for the adsorption in units 104 and 112 to be “equilibrium” PSA that takes advantage of the higher adsorption capacity of methane over nitrogen.

During methane adsorption in units 104 and 112, it is preferred for the temperature within the units to be maintained in the range of from about 40 °F to about 140 °F, more preferably 70 °F to 120 °F. The adsorption pressure in the NRU is preferably maintained in the range of from about one psia to about 200 psia, more preferably from five to 80 psia.

The following Table 1 compares the Material Balances of the various processes set forth in FIG. 1

| Base Case without | Prereated NRU | | |
|---|---|---|---|
| HRM | Feed | CH4 Product | N2 Reject |
| Flow, SCFM | 1000 | 745 | 255 |
| Pressure, psia | 115 | 115 (After | 50 |
| | | compression) | |
| Temperature, °F | 80 | 80 (after | 80 |
| | | cooling) | |
| Composition, % Vol. | | | |
| CH4 | 78.64 | 95 | 30.83 |
| N2 | 20.23 | 4.19 | 67.1 |
| CO2 | 0.5 | 0.68 | 0 |
| O2 | 0.63 | 0.13 | 2.07 |
| CH4 Recovery = 90% | | | |

| | | | Final | |
|---|---|---|---|---|
| | Prereated | Final CH4 | N2 | |
| Case with HRM | NRU Feed | Product | Reject | Recycle |
| Flow, SCFM | 1000 | 801 | 199 | 75 |
| Pressure, psia | 115 | 115 (After | 50 | 115 (After |
| | | compression) | | compression) |
| Temperature, °F | 80 | 80 (after | 80 | 80 (after |
| | | cooling) | | cooling) |
| Composition, | | | | |
| % Vol. | | | | |
| CH4 | 78.64 | 95.04 | 12.73 | 78.64 |
| N2 | 20.23 | 4.2 | 84.65 | 20.72 |
| CO2 | 0.5 | 0.63 | 0 | 0 |
| O2 | 0.63 | 0.13 | 2.62 | 0.64 |
| CH4 Recovery = | | | | |
| 96.8% | | | | |

In the table, both of the methane selective NRUs were running under equilibrium adsorption conditions. The preferred methane selective adsorbent used was equilibrium selective for methane and thus can be used under equilibrium conditions with similar recoveries as shown in Table 1. However, size selected adsorbent can also be used for the first NRU.

As can be seen from the above Table 1, in FIG. 1 a pretreated NRU or landfill feed 101 that contained a mixture of about 80% by volume methane and about 20% by volume nitrogen was introduced into a equilibrium PSA column together with a recycle stream from inlet 122, to form a feed mixture that contained about 80% by volume of methane. It can be seen from Table 1 in connection with FIG. 1 that the single pass recovery without HRM (high recovery NRU module) is 90% by volume. Single pass recovery is defined as methane mol fraction in the gas phase, multiplied by the mols of product per hour divided by the feed methane mol fraction gas phase times the mols of product per hour.

Thus, in connection with FIG. 1, the feed gas to the first NRU is 1000 SCFM times 78.64% CH4=786.4 SCFM of contained CH4) and the product flow is 745 SCFM times 95% methane=707.8 SCFM of contained CH4. Thus the CH4 recovery rate of the first NRU is the CH4 product flow divided by the feed CH4 flow or 707.8 SCFM divided by 786.4 SCFM or 90%.

For the same feed flow rate and conditions (786.4 SCFM of contained CH4 in the feed) the use of the High Recovery Module increases the CH4 product flow to 801 SCFM times 95%=761 SCFM SCFM. The added CH4 recovery by the use of the High Recovery Module is thus 761 SCFM divided by 786.4 SCFM or 96.8% CH4 recovery.

1. A pressure swing adsorption method to produce a high purity methane product stream from a pretreated landfill feed, comprising;
a) sending a pretreated landfill feed containing at least 70% by volume of methane to a first nitrogen rejection unit (“NRU”) containing a methane selective material to adsorb methane and to produce a purged stream enriched in nitrogen;
b) desorbing the methane within the first NRU in a PSA cycle to produce a product stream of methane;
c) sending the purged stream to a second NRU containing a methane selective material to adsorb methane and to produce a second purged stream;
d) desorbing the methane from the second NRU in a PSA cycle to be recycled back to the pretreated landfill feed for the first NRU, to enrich the product stream to contain at least 95% by volume of methane.

2. The method of claim 1 wherein the methane selective adsorbent of steps a) and c) comprises a high aluminum X having a silicon-to-aluminum ratio of about 1, zeolite 13X, carbon or silica gel, molecular sieved activated carbon (“MSC”), crushed activated carbon (“AC-1”), granular activated carbon (“AC-2”), or pelleted activated carbon and mixtures thereof.

3. The method of claim 1, wherein the landfill feed comprises about 80% by volume of methane, less than 2% by volume of water vapor, less than 10% by volume of carbon dioxide, and less than below 1,000 ppm by volume of H₂S.

4. The method of claim 1, wherein the pretreated landfill feed comprises about 5 to 20 mol percent of nitrogen.

5. The method of claim 1, wherein the purged wasted stream from a) containing less than 40% by volume of methane.

6. The method of claim 1, wherein the second waste stream of nitrogen in c) contains less than 20% by volume.

7. The method of claim 1 where a co-current depressurization step is introduced into the first NRU unit to generate the product stream in b).

8. The method of claim 1, wherein a co-current depressurization step is introduced into the second NRU unit to generate the recycled methane stream in d).

9. The method of claim 1 wherein the first and second NRUs operate under conditions of equilibrium selectivity.

10. The method of claim 1, wherein the recycled methane stream in d) contains no more than about 80% by volume of methane.

11. The method of claim 1, wherein the purged waste stream from the first NRU to the second NRU is compressed.

12. The method of claim 1, wherein the recycled stream from the second NRU to the feed for the first NRU is compressed.

13. A pressure swing adsorption system to produce a high purity methane product stream from a pretreated landfill feed comprising:
a) a first nitrogen rejection unit (“NRU”) that contains a methane selective material and is in fluid communication with a pretreated landfill gas feed, such that the first NRU adsorbs methane and allows nitrogen to pass through to produce a first purged stream, and that the adsorbed methane is then desorbed and recovered as a product stream to be removed in a separate outlet;
b) a second nitrogen rejection unit that contains a methane selective material and is in fluid communication with the first NRU to receive the first purged waste stream, such that the second NRU adsorbs methane, and allows nitrogen and other hydrocarbons to pass through, to produce a second purged stream; wherein the second NRU is also in separated fluid communication with the pretreated landfill feed inlet to the first NRU, such that the adsorbed methane within the second NRU is desorbed, recovered and sent to the first NRU together with the pretreated landfill gas.

14. The system of claim 13 wherein the methane selective adsorbent of steps a) and b) comprises a high aluminum X having a silicon-to-aluminum ratio of about 1, zeolite 13X, carbon or silica gel, molecular sieved activated carbon (“MSC”), crushed activated carbon (“AC-1”), granular activated carbon (“AC-2”), or pelleted activated carbon and mixtures thereof.

15. The system of claim 13, wherein the landfill feed comprises about 80% by volume of methane, less than 2% by volume of water vapor, less than 10% by volume of carbon dioxide, and less than below 1,000 ppm by volume of H₂S.

16. The system of claim 13, wherein the landfill feed comprises about 5 to 20 mol percent of nitrogen.

17. The system of claim 13, wherein a compressor is located between the first NRU and the second NRU in b) to compress the purged stream from the first NRU.

18. The system of claim 13, wherein a compressor is located between the second NRU and the inlet that transports the landfill gas feed to the first NRU, to compress the recycled methane stream from the second NRU in b).

19. The system of claim 18, wherein the recycled methane stream contains no more than about 80% by volume of methane.

20. The system of claim 13, wherein the landfill gas feed comprises about 80% by volume of methane, less than 2% by volume of water vapor, less than 10% by volume of carbon dioxide, and less than below 1,000 ppm by volume of H₂S.

21. The system of claim 13, wherein the landfill feed comprises about 5 to 20 mol percent of nitrogen.

22. The system of claim 13, wherein the purged wasted stream from a) containing less than 40% by volume of methane.

23. The system of claim 13, wherein the second waste stream of nitrogen in b) contains less than 20% by volume.

24. A pressure swing adsorption system to produce a high purity methane product stream from a pretreated landfill feed comprising;
a) a first nitrogen rejection unit (“NRU”) that rejects nitrogen using adsorption, membrane or cryogenic technology and is in fluid communication with a pretreated landfill gas feed, such that the first nitrogen rejection unit enriches nitrogen in a rejected stream and enriches methane in a methane product stream;
b) a second nitrogen rejection unit that contains a methane selective material and is in fluid communication with the first NRU to receive the first waste stream, such that the second NRU adsorbs methane, and allows nitrogen and other hydrocarbons to pass through, to produce a second waste stream; wherein the second NRU is also in separated fluid communication with the pretreated landfill feed inlet to the first NRU, such that the adsorbed methane within the second NRU is desorbed, recovered and sent to the first NRU together with the pretreated landfill gas.
